(19) United States
(12) Patent Application Publication
(10) Pub. No.: US 2023/0144748 A1
(43) Pub. Date: May 11, 2023

(54) COMPOSITIONS FOR TREATMENT OF SPINAL CORD INJURY, METHODS AND USES THEREOF

(71) Applicants: Universidade Do Minho, Braga (PT); B'ACIS-ASSOCIAÇÃO CIÊNCIA, INOVAÇÃO E SAÚDE-BRAGA, Braga (PT)

(72) Inventors: António José BRAGA OSÓRIO GOMES SALGADO, Braga (PT); Nuno André MARTINS SILVA, Santa Maria Arnoso (PT); Rita Catarina ASSUNÇÃO RIBEIRO SILVA, São João da Madeira (PT); Eduardo DOMINGOS CORREIA GOMES, Padim da Graça (PT); Luisa PINTO, Soutelo VVD (PT); Sofia Cristina CRAVINO SERRA, Braga (PT); Jorge Miguel CIBRÃO RIBEIRO, Pombeiro de Ribavizela (PT); Susana Isabel GONÇALVES MONTEIRO, Gandra Prd (PT); Andreia Filipa GOMES DE PINHO, Cesar (PT)

(21) Appl. No.: 17/907,723

(22) PCT Filed: Mar. 30, 2021
(86) PCT No.: PCT/IB2021/052630

§ 371 Date: Sep. 29, 2022

(30) Foreign Application Priority Data
Mar. 30, 2020 (EP) 20166914.0
Mar. 30, 2020 (PT) 116206
Mar. 31, 2020 (EP) PCT/EP2020/059158

(51) Int. Cl. A61K 35/28; A61K 9/00; A61P 25/00

(52) U.S. Cl. CPC A61K 35/28; A61K 9/0019; A61P 25/00

(57) ABSTRACT

The present disclosure relates to a method and composition for the treatment or therapy of spinal cord injury, the composition comprising a total secretome, obtainable from mesenchymal stem cells. The composition is suitable for systemic delivery, preferably intravenous systemic delivery.

### TECHNICAL FIELD

The present disclosure relates to a composition for use in the treatment of spinal cord injury comprising a secretome obtainable from adipose tissue-derived stem cells that is administered intravenously or in situ.

### BACKGROUND

Traumatic spinal cord injury (SCI) results in important deficits, negatively impacting the social, working and physiological quality of life of affected individuals. Its consequences are related with its pathophysiology which is characterized by an extended inflammatory response, excitotoxicity, axonal degeneration as well as the formation of a glial scar.

For many years, therapies were based on the administration of methylprednisolone (MP). However, its use is quite controversial, as recent data has failed to demonstrate conclusive beneficial outcomes. Only palliative care is currently available for these patients, therefore posing an urgent need to find other therapeutic strategies.

The secretome of cells is composed of both secreted soluble growth factors and extracellular vesicles. Particularly, the secretome of adipose tissue-derived mesenchymal stem cells (ASCs) contains important neuroregulatory molecules that can impact SCI regeneration, including pigment epithelium-derived factor (PEDF), semaphorins (SEM), cadherins (CDH), Interleukin-6 (IL-6), Glial-derived nexin (GDN), clusterin (CLUS), decorin (DCN) and Beta-1,4-galactosyltransferase 1 (β4Gal-T1).

In an embodiment, the secretome from mesenchymal stem cells (MSCs) isolated from different sources may variate, and with it, their therapeutic potential. The secretome of MSCs derived from bone marrow, adipose tissue or umbilical cord differ in their secretion of neurotrophic, neurogenic, neuroprotective and other factors important to act against distinct pathological processes involved in central nervous system disorders.

Animal models can be used to assess the effect of the SCI treatment with ASCs secretome, preferably the Xenopus laevis model and the mice model. The outcomes can be measured by different means, according to the used model.

In an embodiment, the recovery of a SCI in Xenopus laevis tadpoles after the treatment with ASCs secretome can be assessed by means of a free-swimming test, and immunostaining of anti-acetylated tubulin and anti-GAP-43 in histological sections of the lesioned area.

In an embodiment, mice models can be used to study the effects of ASCs' secretome in SCI. The treatment outcome can be measured by different means preferably, the Basso Mouse Scale (BMS), Von-Frey trial and the recording of ultrasound vocalizations (USVs), as 22 Hz vocalizations are usually emitted by rodents in response to aversive behavioural situations or distress events, such as exposure to predators, pain, startling noises. Beam balance bars are used to study limb coordination after treatment.

The effect of the secretome on the evolution of SCI was already studied using animal models.

Chudickova et al. (1) tested the therapeutic effect of a secretome obtained from Wharton's jelly derived mesenchymal stem cells in a SCI scenario. The injection was given weekly after the lesion for three weeks. The treatment significantly improved axonal sprouting and reduced the number of reactive astrocytes in the lesion area. The secretome solution was injected in situ, specifically by means of an intrathecal injection.

Tsai et al. (2) studied the effect of a secretome obtained from bone marrow stromal cells on SCI. The secretome was administered systemically, via intravenous injection in injured rats. The treatment improved the behavioural recovery and axon densities in the lesion site.

Lu et al. (3) used bone marrow mesenchymal stem cell-derived extracellular vesicles to promote neural recovery after SCI. The systemic administration of the extracellular vesicles, via tail vein injection on injured rats, enhanced neuronal survival and regeneration and improved motor function, as compared to a control solution.

While the cited works show the potential of secretome-based therapies on spinal cord injury, the effect of a systemic delivery of a secretome derived from adipose stem cells in a context of SCI is not mentioned. Bearing in mind the heterogenous composition between the secretome derived from different stem cell sources, the final outcome is not predictable.

These facts are disclosed in order to illustrate the technical problem addressed by the present disclosure.

### GENERAL DESCRIPTION

In an embodiment, the present disclosure relates to an application for ASCs' secretome as a cell-free based therapy for acute SCI.

An acute spinal cord injury (SCI) is caused by trauma to the spinal cord. It is a medical emergency that needs to be treated right away. The severity of symptoms depends on how badly the spinal cord is damaged and where on the cord the injury occurs. The most common causes of spinal cord injuries are motor vehicle accidents.

An aspect of the disclosure comprises the effect of ASCs secretome in SCI. The continuous treatment of injured animals with the secretome revealed a significant motor and sensorial recovery, with improved of locomotor symptoms, as compared to the non-treated animals. The axonal outgrowth and regeneration through the lesion site were also improved after the treatment with the secretome. ASCs' secretome influences the immune response of the injured animal, by reducing the number of inflammatory cells recruited after injury. A reduction of the lesion cavities is also noted.

An aspect of the disclosure relates to the use in the treatment or therapy of an acute spinal cord injury wherein the treatment is for improving the locomotor symptoms, comprising a secretome obtained from mesenchymal stem cells, wherein mesenchymal stem cells are adipose tissue-derived stem cells, wherein the composition is a single dose injectable composition.

The composition of the present disclosure surprisingly shows an effect in the locomotor rehabilitation following acute spinal cord Injury, or in the locomotor relate acute spinal cord injury.

An embodiment comprises the treatment of SCI with ASCs secretome. The secretome of ASCs induces SCI repair through a mechanism that is based on the immunomodulation of the local inflammatory environment, followed by axonal sprouting and neuronal migration.

In an embodiment, when applied in a SCI scenario, the secretome has a neuroprotective and neuroregenerative role, that can be used on the development of therapies for SCI repair. The deficits associated to SCI can then be ameliorated upon application of the ASCs' secretome. Functional repair is achieved with the administration of ASCs' secretome in the acute phase of SCI.

In an aspect of the disclosure, the secretome comprises the unfractioned secretome. Whenever the proteic and/or vesicular fractions (exosomes) are fractioned the therapeutic effect is lost.

In an embodiment, the present disclosure relates to a composition for use in the treatment or therapy of spinal cord injury comprising a secretome obtainable from mesenchymal stem cells. In a particular embodiment, the mesenchymal stem cells are adipose tissue-derived stem cells.

An aspect of the present disclosure comprises the use of the composition related to the present disclosure for the treatment or therapy of acute spinal cord injuries. Particularly, when the injury occurred at cervical, thoracic, lumbar or sacral anatomical level.

In an embodiment, the secretome comprises the proteic and vesicular fractions, and not fractioned parts of it.

In an embodiment, the secretome comprises the following neuroregulatory molecules: pigment epithelium-derived factor (PEDF), semaphorins (SEM), cadherins (CDH), Interleukin-6 (IL-6), Glial-derived nexin (GDN), clusterin (CLUS), decorin (DCN) and Beta-1,4-galactosyltransferase 1 (β4Gal-T1).

In an aspect of the present disclosure, the adipose tissue-derived stem cells are isolated from the abdomen and buttocks, preferably from human adipose tissue obtained through liposuction.

In an embodiment, the adipose-derived stem cells are plastic adherent in standard culture conditions; express CD105, CD73 and CD90 markers; are negative for CD45, CD34, CD14, CD11b, CD79 and HLA-DR markers; and are capable to differentiate to osteoblasts, adipocytes and chondroblasts.

In an embodiment, the composition related to the present disclosure is an injectable composition comprising a basal media for neuronal cell culture, and the secretome obtained from adipose stem cells, preferably human adipose stem cells.

In an embodiment, the composition for use in the treatment or therapy of spinal cord injury is administrated by an intravenous injection or in situ injection.

In a further embodiment, the said composition can be administrated systemically, preferably by intravenous injection using single-dose administration or multi-dose administration plan.

In an embodiment, the dosage of secretome is obtained considering the mass of total protein, which can be quantified by several different means. In the present disclosure, the mass of total protein was measured by means of the Lowry assay. Thence, the total protein concentration is exhibited by a colour change of the sample solution in proportion to protein concentration, which can then be measured using colorimetric techniques.

In an embodiment, the single form consists of an injectable composition, comprising a definitive amount of 50 mg protein/Kg, the whole of which is intended to be administered as a single dose.

In an embodiment, the administration of a first single dose after the spinal cord injury preferably occurs no more than 8 hours after the spinal cord injury, wherein the dosage amount is less than 5 mg protein/Kg/dose.

In an embodiment, the administration of a second and a third dose occurs 24 h and 48 h after the spinal cord injury, wherein the dosage amount is less than 5 mg protein/Kg/dose.

The composition for use according to any of the previous embodiments, comprises the administration of a weekly dose, wherein the dosage amount is less than 5 mg protein/Kg/dose.

In an embodiment, the dosage amount ranges from 2.5-50 mg protein/Kg/dose; preferably 5-30 mg protein/Kg/dose.

The present disclosure also describes a method for obtaining the secretome from adipose-derived stem cell comprising the following steps:

obtaining adipose-derived stem cells;

culturing the cells at a density of 4000 cells/cm² and maintain cells in culture for 24-96 h, preferably 72 h in a suitable medium;

washing the cells with phosphate buffered saline, preferably without Ca²⁺ and Mg²⁺;

washing the cells with basal cell culture media for neuronal cell culture supplemented with a suitable antibiotic, preferably Kanamycin, penicillin, streptomycin or mixtures thereof; more preferably, 1% (v/v) Kanamycin;
culturing adipose stem cells in basal media for conditioning over 24 h;
collecting and centrifuge the cell culture media to remove the debris;
concentrating the collected secretome.

In an embodiment, the method to collect the secretome from a culture of adipose-derived stem cells comprises: culturing adipose-derived stem cells up to passages 5-12 in standard cell culture conditions; plate the cells at a density of 4000 cells/cm² and maintain cells in culture for 24-96 h, preferably 72 h; wash cells 2 to 10 times, preferably 3 to 8 times, with phosphate buffered saline without Ca²⁺ and Mg²⁺; wash 1 to 5 times, preferably 1 to 3 times, with conditioning media, comprising a basal cell culture media for neuronal cell culture and 1% (v/v) Kanamycin; culture adipose stem cell in conditioning media for 24 h; collect and centrifuge the cell culture media to remove the debris; concentrate the collected secretome 50 to 200×, preferably 80-120×, by centrifugation at 1500 to 4000 g, preferably 2500-3500 g.

### BRIEF DESCRIPTION OF THE DRAWINGS

The following figures provide preferred embodiments for illustrating the disclosure and should not be seen as limiting the scope of invention.

FIG. 1: Panel with results regarding an embodiment comprising the therapeutic effects of ASC secretome on Xenopus laevis tadpoles after complete transection on swimming recovery, axonal growth and regeneration. Wherein, (A) depicts the swimming pattern along the experimental time; (B) the quantification of the distance travelled by the animals while swimming at 2, 3 and 5 days post-treatment; (C) gathers representative confocal images of longitudinal cross-sections of Xenopus laevis spinal cord after immunostaining for βIII-tubulin (axonal sprouting, green) and GAP-43 (axonal regeneration, red); (D) plots the quantification of the percentage of βIII-tubulin; (E) illustrates the quantification of GAP-43 positivity. Data represented as mean±SEM; n=15 for locomotor assessment; n=5 for histological evaluation *p<0.05; **p<0.01; ***p<0.001.

FIG. 2: Panel with results regarding an embodiment comprising the recovery of motor and sensorial function of mice with complete spinal cord transection after ASC secretome treatment. Wherein, (A) plots the BMS test performed up to 6 weeks after treatment; (B) is a schematic representation of Von-Frey Trial and corresponding results; (C) plots the recordings of USVs from mice during Von Frey trial, performed at 2 and 6 weeks after ASC secretome treatment; (D) plots the duration of the calls during Von Frey trial, performed at 2 and 6 weeks after ASC secretome treatment. Data is presented as mean±SEM; n=8 (SH), n=7 (NB), n=9 (CM); *p<0.05; **p<0.01; ***p<0.001; ****p<0.0001.

FIG. 3: Illustration of results of an embodiment comprising the therapeutic effects of ASC secretome in the mouse spinal cord, 6 weeks after complete transection. Wherein, representative confocal images of longitudinal cross-sections of mouse spinal cord after immunostaining for Iba-1 (A, neuroinflammation), fluoromyelin (B, lesion cavity), βIII-tubulin (C, axonal growth) and GAP-43 (D, axonal regeneration) are shown. (E) shows the percentage of Iba-1 positivity, (F) the area of lesion cavity, (G) the quantification of the percentage of βIII-tubulin and (H) the percentage of GAP-43, and. Data is presented as Mean±SEM; n=5; *p<0.05; **p<0.01; ***p<0.001; ****p<0.0001.

FIG. 4: Motor recovery evolution of SCI animals treated with ASCs total secretome, proteic fraction or its vesicular fraction. SCI animals exhibit a tendency for having a superior BMS score when treated with total hASC secretome when comparing to animals treated only with the proteic or vesicular fraction. Data presented as mean±SEM (n=9 to CM and vesicles, n=10 to proteic fraction).

FIG. 5: Beam Balance test scoring of SCI animals treated with either ASCs secretome or its proteic and vesicular fraction. SCI animals treated with total ASCs secretome presented more balance and coordination. Data presented as mean±SEM (n=9 for CM, n=8 for protein and n=7 for vesicles, with 2 independent measurement for each animal), ####p<0.0001 and #=different from all conditions.

FIG. 6: Motor recovery evaluation using the BMS test on animals treated with vehicle and ASCs secretome injected locally and systemically. Data is shown as mean±SEM. (*P<0.05).

FIG. 7: Concentration of the pro-inflammatory cytokines IL-6 and IFN-γ.

### DETAILED DESCRIPTION

The present disclosure relates to a composition for use in the treatment or therapy of spinal cord injury comprising a total secretome, obtainable from mesenchymal stem cells, preferably adipose tissue-derived stem cells. The said composition is suitable for systemic delivery, preferably intravenous systemic delivery.

The present disclosure relates to the use of ASCs secretome for the treatment of SCI. The ASCs secretome comprises a proteic and a vesicular fraction, preferably not used as fractioned individual parts.

In an embodiment, the secretome, denoted as conditioned media (CM), was collected from adipose-derived stem cells in passage 5. Cells were plated at a density of 4000 cells/cm² and maintained in culture for 72 hours. Cells were then washed 5 times with phosphate buffered saline (PBS) without Ca²⁺ and Mg²⁺(Invitrogen, USA), and 1 time with the conditioning medium—Neurobasal A Medium supplemented with 1% (v/v) Kanamycin (Invitrogen, USA). After 24 hours of conditioning period in supplemented Neurobasal-A medium, the secretome was collected and centrifuged to remove cell debris. The collected secretome was concentrated 100× using a Vivaspin 20 centrifugal concentrators (MWCO 5 kDa, Sartorius™ Vivaspin™ 20, Germany) at 3000 g, and frozen at −80° C. until further required.

The main components of the secretome of ASCs are listed in Table 1.

TABLE 1
Main components of ASCs' secretome normalized to the
intensity peak of a standard, the recombinant protein
malE-GFP, as measured by mass spectrometry (MS/MS) analysis.

| | | Normalized |
|---|---|---|
| | Abbre- | concentration |
| Protein | viation | to standards |
| Brain-derived neurotrophic factor | BDNF | 0.123 |
| Beta-Nerve growth factor | beta-NGF | 0.06733 |
| Glial cell-derived neurotrophic factor | GDNF | 0.053575 |
| Heparin-binding Epidermal-like growth | HB-EGF | 0.112088 |
| factor | | |
| Insulin-like growth factor-1 | IGF-1 | 0.136405 |
| Matrix metalloproteinase-2 | MMP-2 | 0.05752 |
| Matrix metalloproteinase-3 | MMP-3 | 0.512232 |
| S100 calcium-binding protein B | S100 B | 0.073842 |
| Vascular endothelial growth factor-A | VEGF-A | 0.010158 |
| Granulocyte colony-stimulating factor | GCSF | 0.004744 |
| Interferon gamma | IFNg | 0.109242 |
| Interleukin-10 | IL-10 | 0.020889 |
| Interleukin-1 alpha | IL-1 alpha | 0.045514 |
| Interleukin-6 | IL-6 | 2.0093 |
| Interleukin-8 | IL-8 | 1.721303 |
| Monocyte chemoattractant protein-1 | MCP-1 | 0.876355 |
| Macrophage inflammatory protein-1 alpha | MIP-1 alpha | 0.019186 |
| Transforming growth factor | TGF beta | 0.016577 |
| Tumor necrosis factor-alpha | TNF alpha | 0.07814 |
| Angiogenin | Angio | 0.010964 |
| Epidermal growth factor | EGF | 0.142951 |
| Basic fibroblast growth factor | bFGF | 0.08459 |
| Leptin | Lep | 0.008604 |
| Platelet-derived growth factor-BB | PDGF-BB | 0.017346 |
| Placental growth factor | PLGF | 0.007329 |
| Tissue inhibitor of metalloproteinases-1 | TIMP-1 | 1.193052 |
| Tissue inhibitor of metalloproteinases-2 | TIMP-2 | 1.814304 |
| Thrombopoietin | TPO | 0.038921 |
| Vascular endothelial growth factor-D | VEGF-D | 0.047356 |
| C-X-C chemokine 5 | CXCL-5 | 0.236729 |
| C-X-C chemokine 1/2/3 | CXCL-1/2/3 | 2.361604 |
| Chemokine (C-C motif) 5 | CCL-5 | 0.031711 |
| Protein Deglycase | DJ-1 | 1 |
| Thioredoxin | TRX | 0.5 |
| Cyclophilin A | CYPA | 6. |
| Cyclophilin B | CYPB | 3 |
| Cystatin C | CYSC | 12 |
| Peroxiredoxin-1 | PRDX1 | 1 |
| Serum Albumin | SA | 1 |
| Heat shock 27 kDa protein | HSP27 | 0.5 |
| Galactin-1 | Gal-1 | 10 |
| Pigment epithelium-derived factor | PEDF | 4 |
| Plasminogen activator inhibitor 1 | PAI-1 | 6 |
| Ubiquitin carboxyl-terminal hydrolase | UCH-L1 | 0.5 |
| isozyme L1 | | |
| Plasma protease C1 inhibitor | C1 Inh | 1 |
| Decorin | DCN | 15 |
| Clusterin | CLUS | 8 |
| Cadherin 2 | CADH2 | 1.5 |
| Semaphorin 7A | SEM7A | 1 |
| Glia derived-nexin | GDN | 1 |
| Brain acid soluble protein 1 | BASP-1 | 0.5 |

In an embodiment, the effect of ASCs secretome in promoting spinal cord regeneration after injury is evaluated using the Xenopus laevis animal model. Tadpoles in stage 45-47 do not regenerate spontaneously, and so, any injury to the spinal cord causes irreversible consequences at the motor level. SCI was performed by completely transecting their spinal cord. Immediately after injury, animals received a single injection of ASCs secretome (CM group) through the ependymal canal, rostral to the injury site. Animals inflicted with SCI and injected with Neurobasal-A medium (NB group), or not subjected to SCI and injected with saline solution (SH group) were used as control groups.

In an aspect of this disclosure, the motor recovery of tadpoles in response to secretome treatment was assessed by monitoring animal's free-swimming ability using a motion capturing software, at 2, 3, and 5 days post-injury. Neuronal regrowth and regeneration after treatment is assessed by performing anti-acetylated tubulin and anti-GAP-43 immunostaining, respectively, at 2, 3, and 5 days post-injury for refractive period animal.

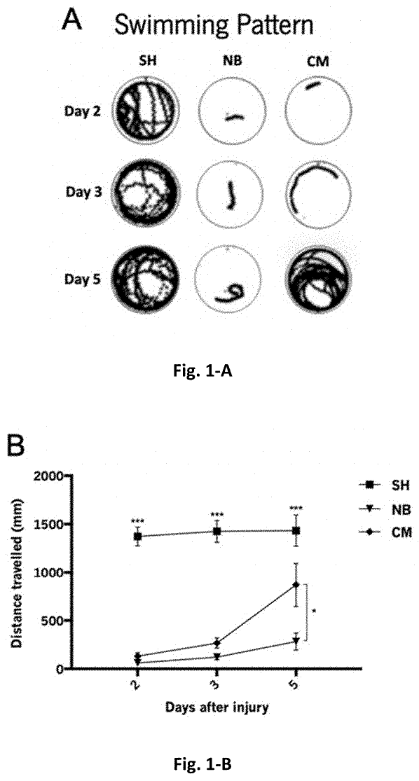

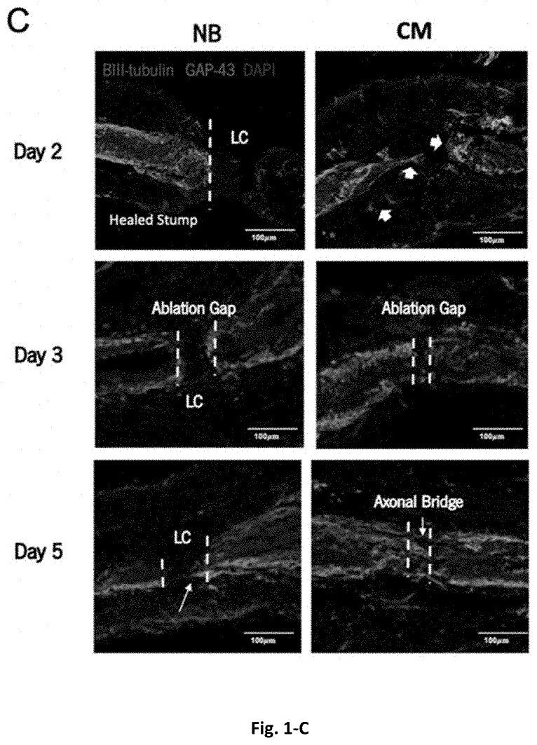

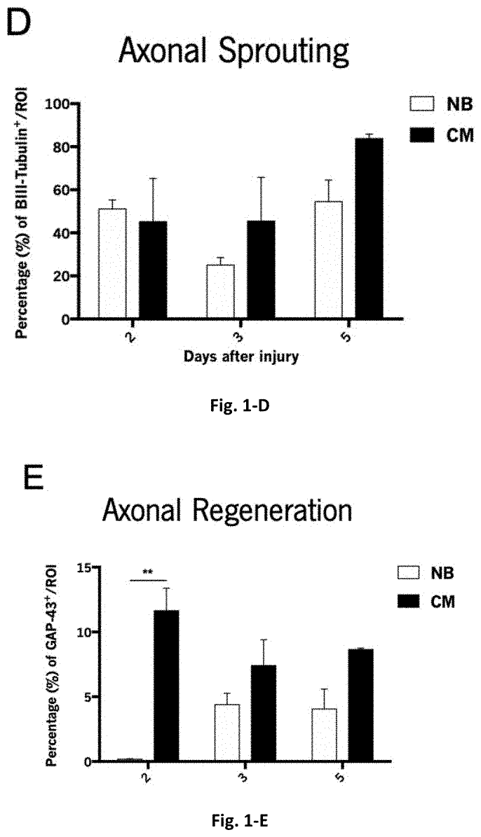

FIG. 1 shows the results of an embodiment of the present disclosure, where the therapeutic effects of ASCs secretome on Xenopus laevis tadpoles is evaluated after complete transection, wherein, A shows the swimming pattern, and B the quantification of the distance travelled by refractory animals at 2, 3, and 5 days post-treatment. Paralysis of all animals was observed during the two initial days post-treatment. On the following days, the ASCs secretome-treated group showed a swimming pattern very similar to healthy animals (SH group), in opposition to NB-treated animals (FIG. 1A). Significant differences in the swimming distances between the ASC secretome-treated groups and the NB-treated group can be detected 5 days post-treatment (*p<0.05; FIG. 1B).

Within the same FIG. 1, in C are represented confocal images of longitudinal cross-sections of Xenopus laevis spinal cord after immunostaining for (axonal sprouting) and GAP-43 (axonal regeneration). Quantification of the percentage of βIII-tubulin and GAP-43 positivity are shown in D and E, respectively. Substantial GAP-43 expressing cells in the lesion core (LC) and both rostral and caudal ends of the spinal cord was observed in the ASCs secretome-treated group (FIG. 1C), 2 days after treatment, but few were observed for the NB-treated group (FIG. 1C, arrow heads). This was confirmed by significant differences in the mean percentage of GAP-43+ cells between the ASC secretome-treated group and the NB-treated group (**p<0.01, FIG. 1D). Moreover, a considerable ablation gap closure and a robust axonal bridge formation was observed in the secretome-treated animals, 3 and 5 days after treatment, respectively (FIG. 1C). Furthermore, increased expression of βIII-tubulin in the LC 5 days post-treatment indicated neuronal regrowth throughout the injury site (FIG. 1E), though no statistical differences were found between groups.

In another embodiment, the impact of ASCs secretome treatment can be evaluated in a rodent animal model, comprising a mouse model after complete transection of the spinal cord. SCI was performed in eight weeks-old female C57BI6/J mice (Charles River, France). Animals were group housed-5 per cage, on corncob bedding with access to food and water ad libitum, and holding rooms were maintained on a 12-hour light/dark cycle. Animals were anesthetized with a mixture of Ketamine 75 mg/Kg) and medetomidine (1 mg/kg). When no reaction to pinch was observed, animals were considered ready for surgery. First, animals were placed under a dissecting microscope. An incision on the skin and dorsal muscles was performed from T2-T10 and the muscles retracted. A laminectomy was performed at the T8 level, and the spinal cord exposed. At this stage, animals were grouped according to the procedure and/or treatment to receive: 1) mice subjected to sham operations-laminectomy but no SCI, injected with Neurobasal-A medium (SH group, n=8); 2) mice subjected to SCI, injected with Neurobasal-A medium (NB group, n=7); and 3) mice subjected to SCI, injected with ASC secretome (CM group, n=9). The spinal cord of NB and CM group animals was totally cut using a microdissection scissor. The complete separation of both ends of the spinal cord was confirmed under the microscope using forceps. Animals were finally closed with Vicryl sutures (Johnson and Johnson, USA). After the surgical procedure, anaesthesia effect was reverted by a single subcutaneous administration of atipamezole (1 mg/Kg, Antisedan/Pfizer, USA). Post-operative care consisting in subcutaneous administration of the analgesic buprenorphine (0.05 mg/Kg, Bupaq, Richter Pharma AG, Austria), the antibiotic enrofloxacin (5 mg/Kg, Baytril/Bayer, Germany), 0.9% (v/v) NaCl and vitamins (Dulphalyte, Pfizer) was then given to every animal. Animals were then kept under heat lamps until recover from anaesthesia. Post-operative care was maintained twice a day for 1-week post-injury. Manual bladder voiding was performed twice a day until animals recover their bladder control completely. The general health of the animals was carefully checked every day for signs of illness and weight loss of the animals, during the time of post-surgery recovery and treatment.

The secretome of ASCs was intravenously administered through animal's tail vein 8, 28, and 48 hours post-injury; and then weekly for a total of 6 weeks. Control animals were administered with Neurobasal-A media after laminectomy (SH group) or SCI (NB group). Secretome was concentrated 100× before injections. Control groups receive concentrated injections of vehicle (basal media for neuronal cell culture). All the transected mice presented complete paraplegia of both hindlimbs 2 days after injury, confirming the complete transection of the spinal cord.

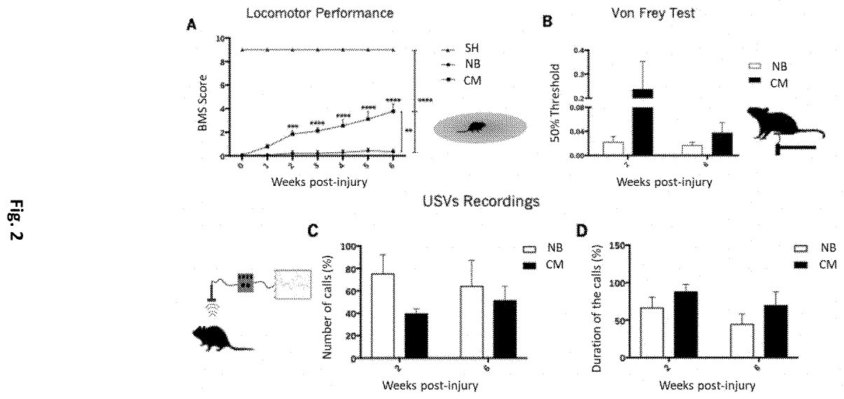

In an aspect of this disclosure, locomotor analysis of mice treated with ASCs secretome showed a significant clear and progressive motor recovery. FIG. 2A depicts the result of Basso Mouse Scale (BMS) score of the different test groups. Motor recovery started at 2 weeks post-treatment and did not plateau until the end of the experiment, 6 weeks post-treatment. At this time-point, the CM group presented the ability to perform coordinated plantar stepping, while the NB-treated group only presented slight movement of ankles.

In an embodiment, the locomotor improvements of the secretome-treated animals were accompanied by an improvement of sensitivity from 2-to 6-weeks post-treatment, inferred by the higher threshold of the animal's response to Von Frey filaments in the CM-group (FIG. 2B). Although no statistical differences were found between groups at both time-points, CM group show a trend of recovery of the sensorial function, when compared to NB group. Interestingly, no pain or discomfort was associated with Von Frey stimuli, inferred by the lower number (FIG. 2C) and higher duration (FIG. 2D) of negative vocalizations (22 Hz) during the time of the mechanical stimulus to these animals (FIG. 2D).

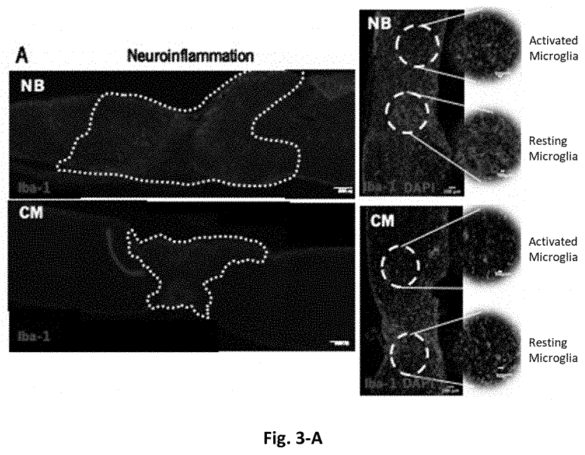

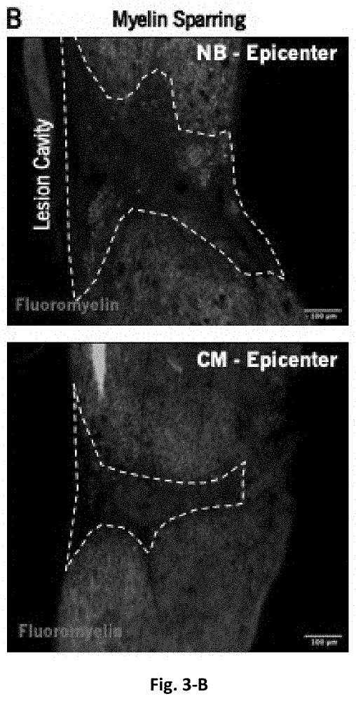

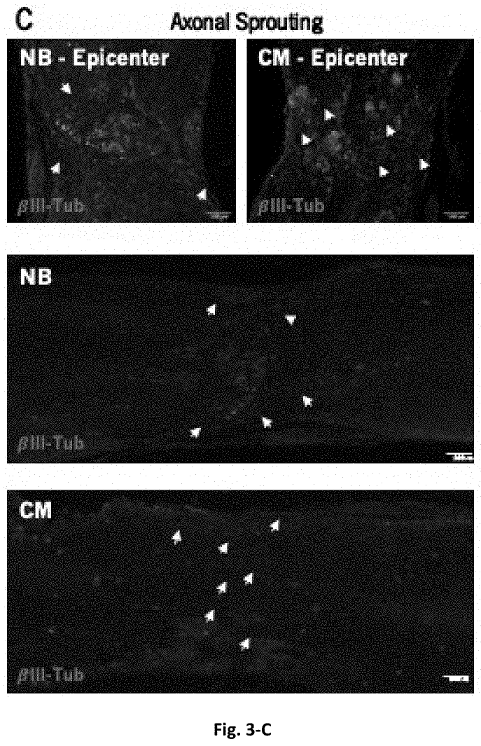

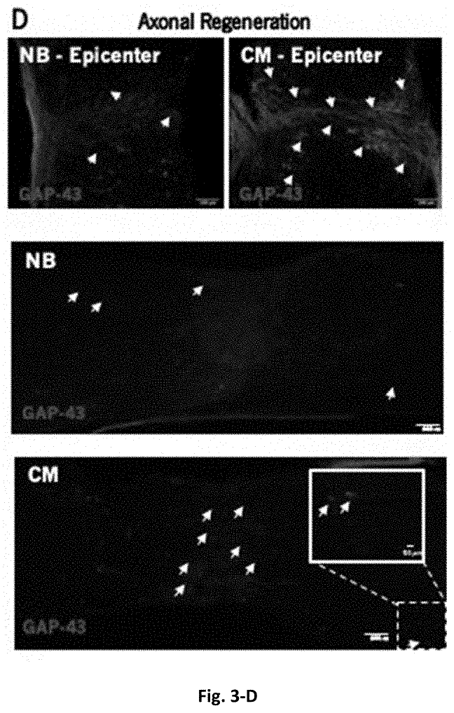

In an aspect of the disclosure, the motor and sensorial recovery of SCI animals after ASCs secretome treatment is explained by the observed axonal elongation in the spinal cord of these animals from the cut ends of the spinal cord to the epicentre of the lesion, at 6 weeks post-treatment, inferred by βIII-tubulin positivity (FIG. 3C, 3G).

An aspect of the disclosure comprises the effect of ASCs secretome on the regenerative process, wherein the exogenous supply of ASCs secretome prolongs the regenerative process up to 6 weeks post-injury. A significant expression of GAP-43 by axons in the lesion epicentre, with some fibres also coursing rostral and caudally to the injury site, for the secretome-treated animals (CM group), was noticed in comparison to the NB-treated mice, at 6 weeks post-treatment (FIG. 3D, 3H). Along with axonal sprouting and regeneration, decreased lesion cavities were also observed for the secretome-treated animals, in comparison to the NB-treated animals (FIG. 3B, 3F).

In an embodiment, the ASCs secretome treatment attenuates the inflammatory response of microglial cells on the injured animals. Clear differences in Iba-1 expression are noticed between the secretome- and NB-treated animals at 6 weeks post-treatment (FIG. 3A), with the CM group revealing significant decreased Iba-1⁺ activated inflammatory cells in the spinal cord than the NB group, and significant increased Iba-1⁺ resting cells (FIG. 3A, 3E).

In an embodiment, the ASCs secretome is constituted by soluble factors, micro vesicles, exosomes and apoptotic bodies.

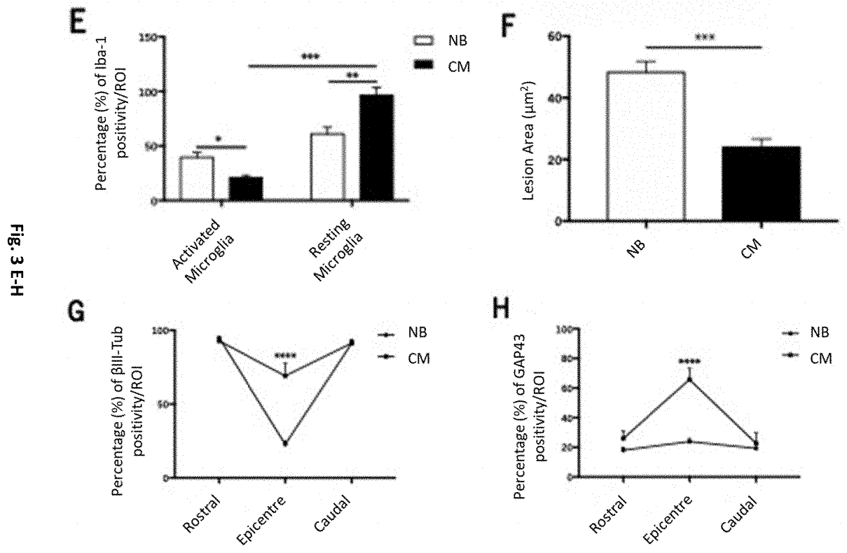

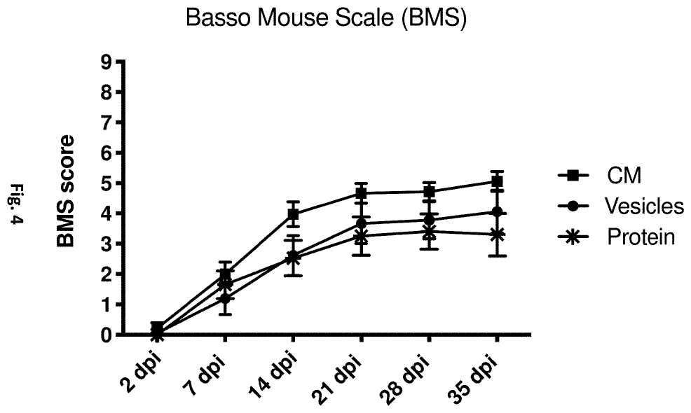

In an aspect of the disclosure, the therapeutic effect is only noticed when the total ASCs secretome is used. Along time, it is observed a motor function recovery with animals starting from no movement of the hindlimbs to exhibit frequent plantar stepping with some coordination, when treated with total ASC secretome (CM). In contrast, animals treated with the different fractions (proteic or vesicular) could only do frequently plantar stepping (FIG. 4). Coordination was also analysed using the beam balance bars, revealing a statistically significant difference between animals treated with total ASCs secretome and animals treated only with the proteic or vesicular fraction (FIG. 5).

In another embodiment, it was compared the therapeutic effect of the secretome when injected locally or via intravenous injection. A complete compression of the spinal cord was used for the present embodiment. Briefly, animals were anesthetized with a mixture of Ketamine (75 mg/Kg) and medetomidine (1 mg/Kg). When no reaction to pinch was observed, animals were considered ready for surgery. First, animals were placed under a dissecting microscope. An incision on the skin and dorsal muscles was performed from T2-T10 and the muscles retracted. A laminectomy was performed at the T8 level, and the spinal cord exposed. At this stage, animals were grouped according to the procedure and/or treatment to receive: 1) mice subjected to SCI, injected with Neurobasal-A medium (NB group, n=6); 3) mice subjected to SCI, injected with ASC secretome in the spinal cord (CM group, n=4); 4) mice subjected to SCI, injected with ASC secretome intravenously (CM group, n=9). The spinal cord of NB and CM group animals was compressed for 10 s using a compression clip. Animals were finally closed with Vicryl sutures (Johnson and Johnson, USA). After the surgical procedure, anaesthesia effect was reverted by a single subcutaneous administration of atipamezole (1 mg/Kg, Antisedan/Pfizer, USA). Post-operative care consisting in subcutaneous administration of the analgesic buprenorphine (0.05 mg/Kg, Bupaq, Richter Pharma AG, Austria), the antibiotic enrofloxacin (5 mg/Kg, Baytril/Bayer, Germany), 0.9% (v/v) NaCl and vitamins (Dulphalyte, Pfizer) was then given to every animal. Animals were then kept under heat lamps until recover from anaesthesia. Post-operative care was maintained twice a day for 1-week post-injury. Manual bladder evacuation was performed twice a day until animals recover their bladder control completely. The general health of the animals was carefully checked every day for signs of illness and weight loss of the animals, during the time of post-surgery recovery and treatment.

In an aspect of the present embodiment, intravenous administration was based on three injections at the tail vein, 8 h, 28 h, 48 h after injury, followed by weekly injections (7 days apart) until animal were euthanized. Secretome was concentrated 100×before injections. For spinal cord tissue administration, secretome was directly injected once in the spinal cord tissue upon injury was performed.

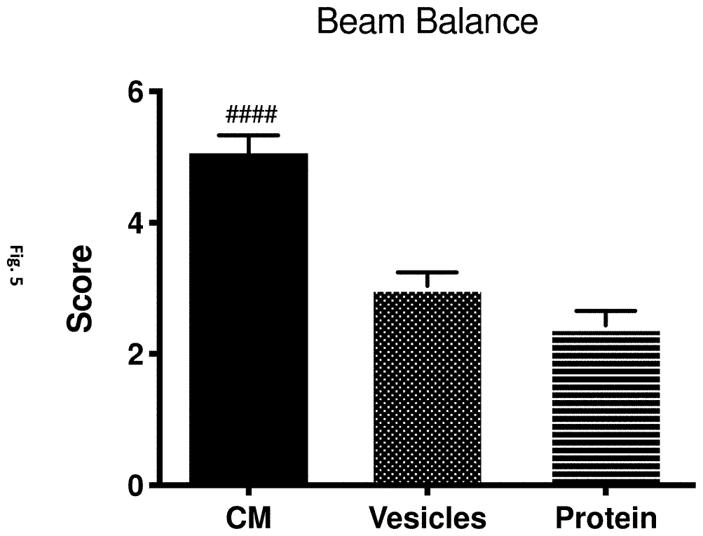

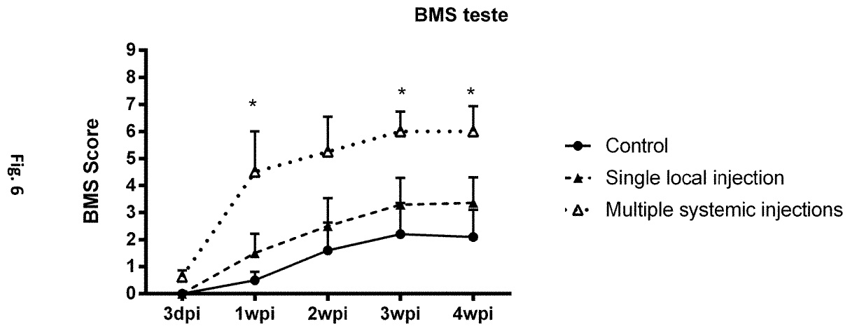

Locomotor evaluation using the BMS score started 3 days after injury and was subsequently repeated once a week for 4 weeks to assess the level of functional locomotor recovery (FIG. 6). Three days after the compression injury, all groups demonstrate decreased locomotor function, control and local treatment (group score=0) and systemic treatment with a score of 0.625±0.239, indicating that the hindlimbs function is severe impaired as only slight movement of the ankle was detected. The control group, control medium injection, showed a spontaneous locomotor recovery over time, stabilizing after 3 weeks post-injury reaching a maximum score of 2.1±1, which translate in extensive ankle movement. Local treatment with secretome demonstrates a recovery slightly superior to the control group, stabilizing at week 3 and reaching a maximum score of 3.35±0.94 (Plantar placing of the paw with or without weight support, or occasional, frequent or consistent dorsal stepping but no plantar stepping). In the systemic-treated group, BMS score achieved a maximum score of 6±0.93, which represents frequent or consistent plantar stepping, some coordination, paws parallel at initial contact, or frequent or consistent plantar stepping, mostly coordinated, paws rotated at initial contact and lift off. Furthermore, there is a significant statistical difference between this group and the control at 1, 3- and 4-weeks post injury (p<0.05). The systemic group also display a higher BMS score than the local treatment (FIG. 6).

In an embodiment, the concentration (pg/ml) of pro-inflammatory-IL-6, IFN-cytokines in the blood serum of SCI mice was assessed using multiplex-based ELISA

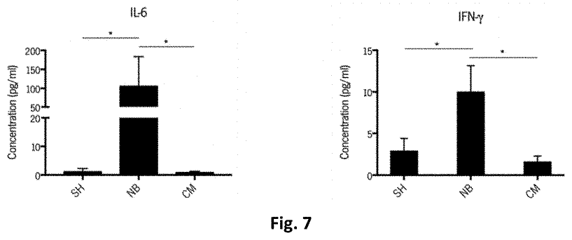

(FIG. 7). CM-treated animals showed decreased levels of pro-inflammatory cytokines, and increased levels of anti-inflammatory cytokines, when compared to NB- and SH-treated groups. Mean±SEM; n=7 for SH and CM groups; n=4 for NB group; *p<0.05.

In an embodiment, to obtain MSCs without serum supplementation and without other products of animal origin, previously generated iPSCs lines (NRC-2H, NRC-4J, NRC-5H) were expanded in a feeder-free system in mTeSR™ medium onto vitronectin-coated 6-well tissue culture plates (all products from STEMCELL Technologies, Canada). Afterwards, STEMdiff™ Mesenchymal Progenitor Kit (STEMCELL technologies) was used to differentiate iPSCs into MSC-like cells, according to manufacturer's instructions. Briefly, iPSCs were dissociated into a single cell suspension after incubation with Gentle Cell Dissociation Reagent (STEMCELL Technologies, Canada) for 8-10 min. After centrifugation, 500 000 cells were seeded on vitronectin-coated 6-well tissue culture plates in mTeSR™ medium containing 10 μM of ROCK Inhibitor Y-27632 (Selleck Chemicals, USA). After 2 days and for the next 6 days, cells were kept in STEMdiff™-ACF Mesenchymal Induction Medium, with daily medium changes. At day 6, cells were passaged with Gentle Cell Dissociation Reagent (STEMCELL Technologies, Canada) into a new 6-well tissue culture plate in MesenCult™-ACF Plus Medium. From passage 2 onwards, cells were passaged at 2 000 cells/cm² using Animal Component-Free Cell Dissociation Kit (STEMCELL Technologies, Canada).

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above described embodiments are combinable.

The following claims further set out particular embodiments of the disclosure.

The following references, should be considered herewith incorporated in their entirety:
1. M. Chudickova, I. Vackova, L. Machova Urdzikova, P. Jancova, K. Kekulova, M. Rehorova, K. Turnovcova, P. Jendelova, and Sarka Kubinova., “The Effect of Wharton Jelly-Derived Mesenchymal Stromal Cells and Their Conditioned Media in the Treatment of a Rat Spinal Cord Injury”, Int. J. Mol. Sci. 2019, 20(18), 4516; httpslidoi.org/10.3390/ijms20184516
2. M. Tsai, D. Liou, Y. Lin, C. Weng, M. Huang, W. Huang, F. Tseng, and H. Cheng, “Attenuating Spinal Cord Injury by Conditioned Medium from Bone Marrow Mesenchymal Stem Cells”, J. Clin. Med. 2019, 8(1), 23; https://doi.org/10.3390/jcm8010023
3. Y. Lu, Y. Zhou, R. Zhang, L. Wen, K. Wu, Y. Li, Y. Yao, R. Duan, and Y. Jia, “Bone Mesenchymal Stem Cell-Derived Extracellular Vesicles Promote Recovery Following Spinal Cord Injury via Improvement of the Integrity of the Blood-Spinal Cord Barrier”, Front. Neurosci., 12 Mar. 2019; httpslidoi.org/10.3389/fnins.2019.00209

1. A method of treating a patient with an acute spinal cord injury, the method comprising administering to the patient a composition comprising a secretome obtained from mesenchymal stem cells, wherein the mesenchymal stem cells are adipose tissue-derived stem cells, wherein the composition is a single dose injectable composition and wherein the composition improves locomotor symptoms in the patient.

2. The method of claim 1, wherein the single dose comprises an amount of less than 5 mg/dose of secretome proteins.

3. The method of claim 1, wherein the composition is administrated by an intravenous injection or an in situ injection.

4. (canceled)

5. The method of claim 1, wherein the single dose is administered 8 hours after the spinal cord injury.

6. The method of claim 1, further comprising administering second and third doses of the composition, wherein the second and third doses are administered 24 h and 48 h, respectively, after the spinal cord injury.

7. The method of claim 1, wherein the composition is administered to the patient in a weekly dose.

8. The method of claim 1, wherein the composition further comprises a carrier selected from the group consisting of: standard cell culture media Neurobasal, Neurobasal A, DMEM, alpha-MEM and DMEM/F12 cell culture media.

9. The method of claim 1, wherein the secretome comprises proteic and vesicular fractions.

10. The method of claim 1, wherein the secretome comprises the following neuroregulatory molecules: pigment epithelium-derived factor (PEDF), semaphorins (SEM), cadherins (CDH), Interleukin-6 (IL-6), Glial-derived nexin (GDN), clusterin (CLUS), decorin (DCN) and Beta-1,4-galactosyltransferase 1 (β4Gal-T1).

11. The method of claim 1, wherein the source of adipose tissue-derived stem cells is selected from the abdomen and buttocks.

12. The method of claim 1, wherein the adipose tissue-derived stem cells are:
plastic adherent in standard culture conditions;
express CD105, CD73 and CD90 markers;
are negative for CD45, CD34, CD14, CD11 b, CD79 and HLA-DR markers; and
are capable to differentiate to osteoblasts, adipocytes and chondroblasts.

13. The method of claim 1, wherein the spinal cord injury occurred at cervical, thoracic, lumbar or sacral anatomical level.

14. The method of claim 1, wherein the composition further comprises a basal media for neuronal cell culture and wherein the secretome is obtained from human adipose stem cells.

15. The method of claim 1, wherein the composition is for single-dose administration or a multi-dose administration.

16. A method for obtaining a secretome from adipose-derived stem cells comprising the following steps:
obtaining the adipose-derived stem cells;
culturing the adipose-derived stem cells at a density of 4000 cells/cm² and maintaining the adipose-derived stem cells in culture for 24-96 h, in a suitable medium;
washing the adipose-derived stem cells with phosphate buffered saline;
washing the adipose-derived stem cells with basal cell culture media for neuronal cell culture supplemented with a suitable antibiotic;
culturing the adipose stem cells in the basal cell culture media over 24 h;
collecting and centrifuging the basal cell culture media to remove debris; and
concentrating the collected secretome.
